(19) United States
(12) Patent Application Publication
(10) Pub. No.: US 2009/0247581 A1
(43) Pub. Date: Oct. 1, 2009

(54) Organic Compounds

(76) Inventors: Mahavir Prashad, Montville, NJ (US); Yugang Liu, Bridgewater, NJ (US); Bin Hu, Green Brook, NJ (US); Michael J. Girgis, Montville, NJ (US); Frank Schaefer, Rheinfelden (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/085,056

(22) PCT Filed: Nov. 16, 2006
(86) PCT No.: PCT/US2006/060980

§ 371 Date: May 14, 2008

Related U.S. Application Data
(60) Provisional application No. 60/737,633, filed on Nov. 17, 2005.

(51) Int. Cl. C07D 211/26; A61K 31/445

(52) U.S. Cl. 514/317; 546/229

(57) ABSTRACT

The present invention relates to a process for the synthesis of (S)-2′[2-1-(methyl-2-piperidyl) ethyl] cinnamanilide (I) or salts or pharmaceutically acceptable prodrugs thereof:

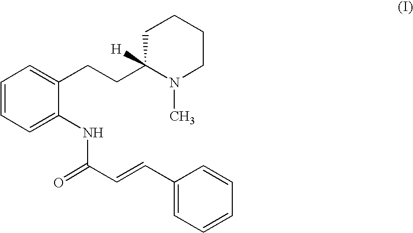

The present invention relates to a process for the synthesis of (S)-2′[2-1-(methyl-2-piperidyl) ethyl] cinnamanilide (I) or salts or pharmaceutically acceptable prodrugs thereof:

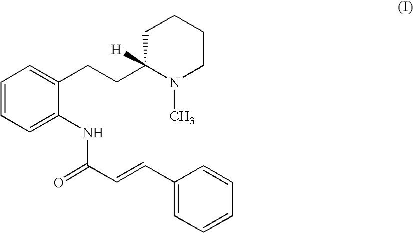

The present invention also relates to a process for providing an intermediate useful in the preparation of the compound (I).

The compound (I) is well described in the art. The compound (I) may be used as a 5-HT₂ antagonist, for example. Furthermore, compound (I) may be used as a pharmaceutical agent for treating 5-HT₂-related diseases such as haemorrhoids, for example.

### DETAILED DESCRIPTION OF THE INVENTION

In one aspect, the invention relates directly or indirectly to the manufacture of compounds of formula I, as illustrated below in Scheme 1:

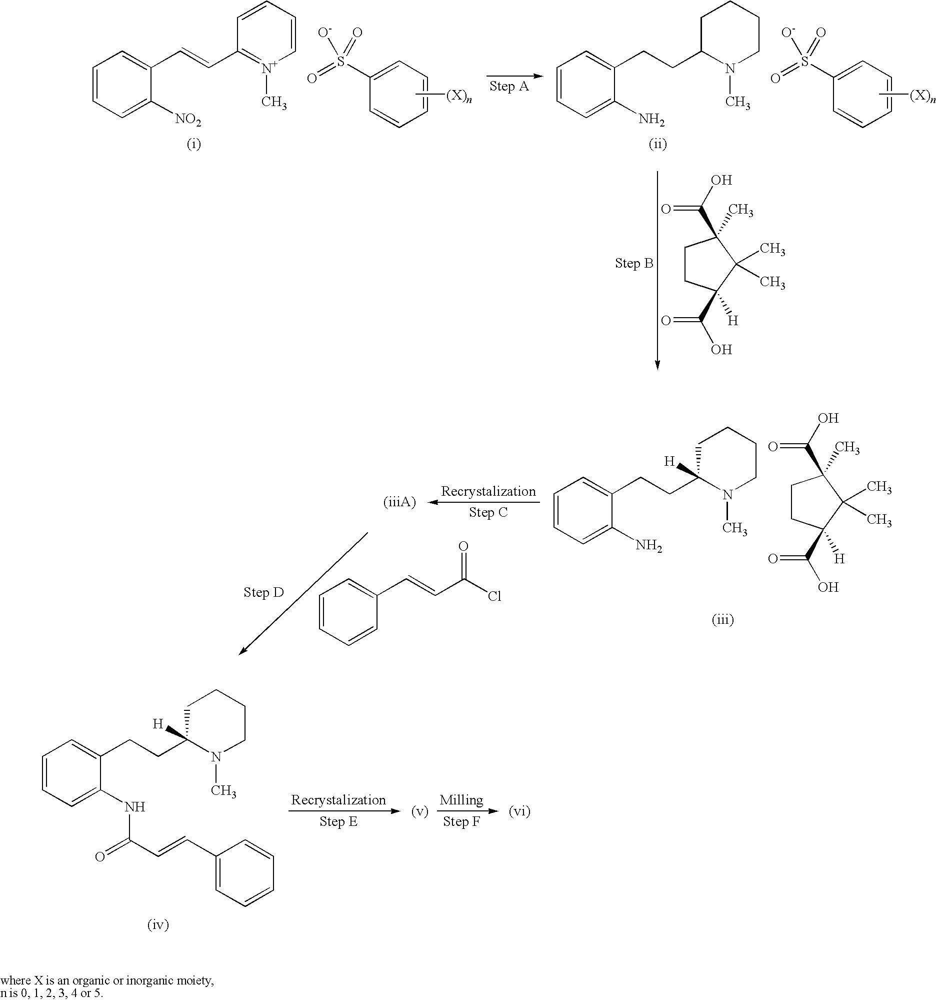

The benzene sulphonate ion (j) will next be described in more detail by way of example:

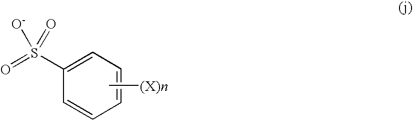

In one class of compounds, X is selected from —OH, NR^{c}R^{d}, halogen, C₁, C₂, C₃ or C₄ alkyl, C₁, C₂, C₃ or C₄ haloalkyl, C₁, C₂, C₃ or C₄ alkoxy, C₁, C₂, C₃ or C₄ alkenyl.

R^{c} and R^{d} are each independently selected from hydrogen, —OH, C₁, C₂, C₃ or C₄ alkyl, C₁, C₂, C₃ or C₄ haloalkyl, C₁, C₂, C₃ or C₄ alkoxy, C₁, C₂, C₃ or C₄ alkenyl.

Halogen may be selected from chloro, fluoro, bromo and iodo, e.g. chloro or fluoro.

The organic moieties, for example C₁, C₂, C₃ or C₄ alkyl, C₁, C₂, C₃ or C₄ haloalkyl, C₁, C₂, C₃ or C₄ alkoxy, C₁, C₂, C₃ or C₄ alkenyl, may be substituted or unsubstituted.

In a further class of compounds, n is 1.

A preferred substituent X is alkyl. In particular, X is methyl.

In a particularly preferred embodiment, the benzene sulphonate ion (j) has a substituent X meta or para to the SO₃ group. Particularly preferred is para. In this embodiment, there is most preferably a single substituent, e.g. alkyl as previously described.

Therefore a preferred reaction scheme for step A is set out below:

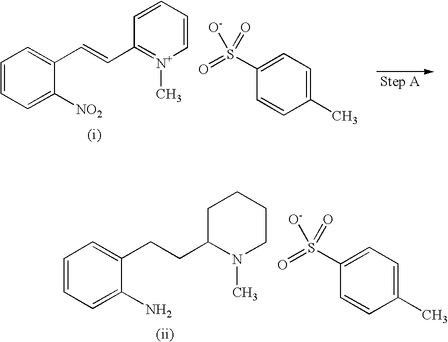

Included in the invention are:

(i) the method of step B;
(ii) the method of step C;
(iii) the method of step D;
(iv) the method of step E;
(v) any method comprising one of (i), (ii), (iii), and (iv);
(vi) any method comprising a combination of two or more of (i), (ii), (iii) and (iv), e.g. (i) followed by (ii) or (ii) followed by (iii) or (iii) followed by (iv).

In one aspect of the present invention, there is provided a process for isolating an isomer of the free base (III):

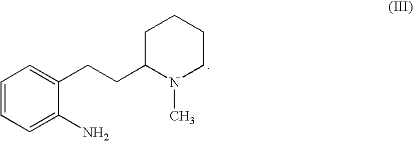

In a particular aspect of the present invention, the isomer (IV) may be isolated:

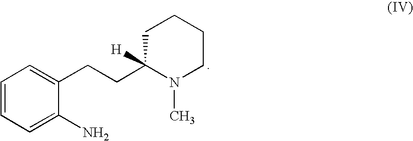

The isomer (IV) may be isolated as a salt. In particular, the isomer (IV) may be isolated as a salt of a resolving agent. The isomer (IV) is the (S)-isomer of the free base (III).

The invention therefore includes a method comprising:

(a) providing compound X in the form of a salt of formula (ii):

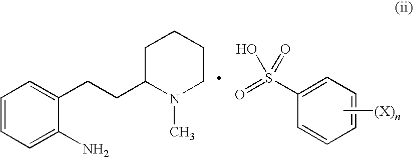

where X is an organic or inorganic moiety; and
n is 0, 1, 2, 3 or 4.
   (b) resolving the isomers of compound (III) with a resolving agent.

The invention also includes:

(b1) converting the salt (ii) into its free base, namely compound (III); and
(b2) contacting the free base with the resolving agent.

The free base of compound (ii) may be optionally isolated before being contacted with the resolving agent. Thus in one embodiment the compound (ii) is isolated and in another embodiment, the compound (ii) is not isolated.

The free base of the compound (ii) may exist in either the (R)- and/or (S)-isomer.

The resolving agent may be an acid, for example a camphoric acid. In particular, the resolving agent is a d-camphoric acid, e.g.:

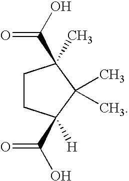

In a particular aspect of the present invention, the free base (III) forms a salt between one of the (R)- and (S)-isomers of the free base (III) and the resolving agent.

In particular, the (S)-isomer (IV) of the free base (III) forms the salt with the resolving agent. The salt of the (S)-isomer is preferably separatable from the resulting reaction mixture, e.g. as a solid, such as a precipitation product. In other words, the invention includes methods in which the resolving agent combines with the base to form a salt substantially insoluble in the reaction medium.

The invention includes a method for resolving the (R)- and (S)-isomers of compound (III) using a resolving agent such as, for example, a d-camphoric acid, wherein the starting material is a benzene sulphonate salt of compound (III). The benzene sulphonate may be substituted or unsubstituted

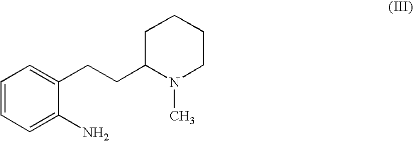

According to a further aspect of the present invention, the isomer (IV) in either or both salt or free base form is an intermediate in the synthesis of compound (I).

The reaction steps of scheme 1 will now be described in more detail below by way of example. In the following description, the skilled person will appreciate that equivalent procedures may be used to those described, e.g. an alternative agitation method may be used in place of stirring, for example.

The term isolated as used herein may be taken to mean separated from and may or may not include physical isolation. An isolated product may not be 100% purity and may contain amounts of other products. Preferably, isolation provides a product with a purity sufficient to enable the process to meet the requirements for pharmaceutical development.

For the procedures for the measurements for LOD (Loss On Drying), the skilled person is referred to the relevant Examples herein.

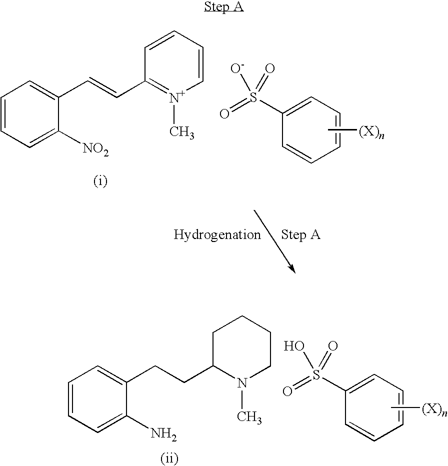

A vessel has an inert atmosphere, achieved by, for example, pressurising with nitrogen to 4.5 bar, then depressurising to 1 bar and repeating this pressurisation/depressurisation four times. The compound may then be added to the vessel. After addition of the aforementioned product, the vessel may then be pressurised/depressurised a further four times with nitrogen. Then, a catalyst, e.g. polor Pt in the presence of carbon, for example 10% Pd/C is added to the vessel. The vessel may then once again be pressurised and depressurised four times with nitrogen. Then, an alcohol, e.g. methanol may be added. Then, the vessel may once again be pressurised/depressurised four times with nitrogen. Each pressurisation step may be up to 5 bar, for example up to 4.5 bar. The depressurisation may be down to 1 bar.

The vessel may then be stirred, at a rate sufficient to obtain at least partial suspension of the catalyst, e.g. full suspension of the catalyst, for example at a rate of about 450 rpm, and the temperature may be set at 25 to 35° C., for example 30° C. The temperature may be allowed to equilibrate at about 30° C. Stirring may then be stopped once equilibrium has been reached. The nitrogen may then be replaced with hydrogen by pressurising the vessel with hydrogen to 4.5 bar and then depressurising to 1 bar. The pressurisation/depressurisation cycle may be carried out a further four times. The agitator (or stirrer) may be turned off during hydrogen introduction to prevent hydrogen reaction from occurring at an early stage. After the final depressurisation, the vessel may be pressurised to about 3-5 bar, for example about 5 bar, typically 5.2 bar, by the introduction of nitrogen for example, and agitated, at a rate sufficient to obtain at least partial suspension of the catalyst, e.g. full suspension of the catalyst, for example at a rate of about 450 rpm.

The agitation may serve to start the reaction. The initial reaction is exothermic, giving a maximum heat evolution rate of about 35 W/kg (except for a short-lived spike with a maximum of about 50 W/kg). The reaction may be detected by hydrogen uptake and heat evolution. The hydrogenation process may be carried out at about 30° C. and about 5.2 bar for about 5-10 hours, for example 7-8 hours, typically 7.2 hours. Then, the vessel may be depressurised to 1 bar and purged with nitrogen, by pressurising to 4.5 bar and depressurising as aforementioned. A total of five pressurisation/depressurisation cycles may be conducted. The reactor may then be emptied and rinsed with an alcohol, e.g. methanol. The e.g. methanol rinse may then be combined with the reaction mixture. The final batch may then be filtered e.g. over a pad of celite. The e.g. celite pad may then be washed with further alcohol e.g. methanol and the filtrate combined. The filtrate may then be distilled at an internal temperature of 30 to 50° C., for example 35 to 45° C. jacket temperature 65 to 75° C.) under reduced pressure (80 to 160 mbar) to a volume of about one third. To the reduced-volume filtrate may be added a peroxide-free alcohol, e.g. 2-propanol. The reaction mixture may then be distilled at an internal temperature of 30 to 50° C., e.g. 35 to 45° C. (jacket temperature 65 to 75° C.) under reduced pressure (80 to 160 mbar) to approximately one third. The reduced-volume mixture is then heated to an internal temperature of 40 to 80° C., e.g. 50 to 70° C., typically 60±5° C. over a period of about 20 minutes and then an acetate, for example an alcohol acetate, typically isopropyl acetate may be added over a period of about 20 minutes while maintaining the internal temperature at about 55 to 65° C. The reaction mixture may then be cooled to an internal temperature of about 40±5° C. over a period of about 20 minutes and the mixture seeded with a small amount of the product. The resulting mixture, e.g. suspension, may be cooled to an internal temperature of about 20±5° C. over a period of about 1 hour and stirred at this temperature for an additional 4 hours for example. The resulting solid may then be collected by filtration and optionally washed with a solvent, for example a mixture of solvents, which may be a mixture of an alcohol and an acetate, for example an alcohol and alcohol acetate, typically 2-propanol and isopropyl acetate. The solvent is preferably in a mixture of alcohol:acetate of 1:2 v/v. The solid is optionally washed two times. The solid may then be dried under reduced pressure (15 to 49 mbar) at approximately 60° C. The drying is completed once the LOD is less than 1%.

The preferred hydrogenation conditions include 10% Pt/C (65% wet) with 2.5% loading.

The hydrogenation reaction of step A is carried out at a high pressure, which may provide a route for higher selectivity for the desired product.

The reaction temperature is maintained at a relatively low level in order to favour the formation of the desired product. It was found by the present inventors that increasing temperature increased by-products, in particular a products such as A and B, below.

The reaction is preferably agitated e.g. stirred, at a rate of between 100 and 300 rpm, for example 150 to 250 rpm, typically 170 to 200 rpm. The rate of agitation may be directly related to mass transfer.

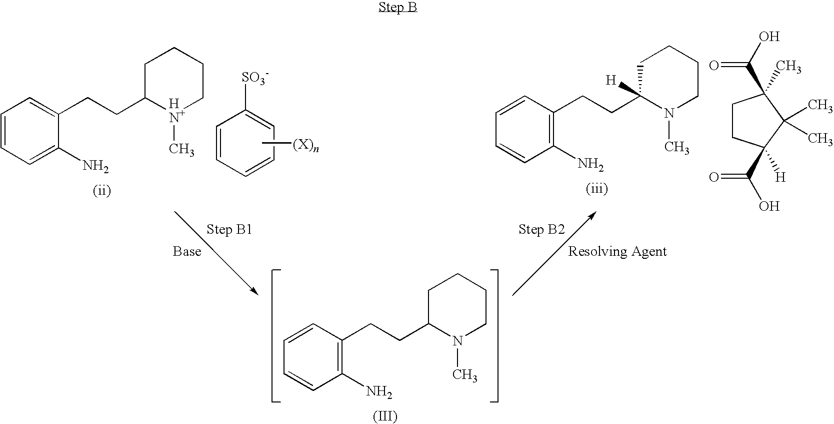

A benzenesulfonate salt of 2-[2-(1-methyl-2-piperidinyl)ethyl]-benzenamine (ii), for example, a methylbenzenesulfonate salt, typically 4-methylbenzenesulfonate may be added to a solvent, for example an acetate, for example an alkyl acetate, typically isopropyl acetate or ethyl acetate. The benzenesulfonate salt may have a stoichiometry of 1:1.

The solvent may also be any aromatic hydrocarbon, for example benzene, alkylbenzene, such as toluene or xylene or aromatic naphtha. In particular, toluene may be chosen as an alternative solvent.

The resulting reaction mixture may be stirred at 15 to 30° C., for example 18 to 27° C., typically 20 to 25° C. The reaction mixture may be under an inert atmosphere, for example under nitrogen. Preferably, the reaction mixture is under nitrogen conditions. Then, to the reaction mixture, may be added a solution of a base, e.g. an alkali metal hydroxide, particularly sodium hydroxide (50-200 mmol, typically about 100 mmol in water) may be added over a period of about 3 to 10 minutes, for example about 5 minutes, whilst maintaining an internal temperature of 15-30° C., such as 20 to 25° C., for example. The resulting reaction mixture, e.g. suspension, may then be stirred until all of the solid is dissolved. The resulting two-phase reaction mixture may then be separated. The organic layer may be removed and the aqueous layer may be extracted with a solvent, for example an acetate, such as an alkyl acetate, for example which may be typically isopropyl acetate. The resulting organic layers may then be combined and optionally washed with water. The resulting two-phase mixture may then be separated and the organic layer reduced in volume, for example under vacuum (10-110 mbar, for example 20-100 mbar) at an internal temperature of between 10 and 50° C., for example 20 and 40° C. (external temperature 30 to 60° C., for example). The resulting reduced-volume organic layer, may be of a volume of about 10 to 50 ml, for example 20 to 30 ml. Then, to the reduced-volume organic layer may be added an alcohol, for example propanol, typically 2-propanol. The reaction mixture may then be concentrated, for example under vacuum (10 to 110 mbar, for example 20 to 100 mbar) at an internal temperature of 10 to 50° C., for example 20 to 40° C. (external temperature 30 to 60° C.). The resulting reduced-volume product may be of a volume of from 10 to 40 ml, for example 20 to 30 ml. Then, the resulting reduced-volume product may then be treated with a further amount of alcohol and then may be further concentrated, for example under vacuum (10 to 110 mbar, for example 20 to 100 mbar) at an internal temperature of 10 to 50° C., for example 20 to 40° C. (external temperature 30 to 60° C.). The resulting reduced-volume product may be reduced to a volume of approximately 20 to 30 ml. Then, the resulting reduced-volume product may be treated with a further alcohol, for example a propanol, typically 2-propanol to obtain a solution of the product ((±)-2-[2-(1-methyl-2-piperidinyl)ethyl]-benzenamine) (iii) in 2-propanol.

The amount of alcohol, e.g. 2-propanol, added to the aforementioned reduced-volume product may be adjusted to provide a specific concentration of the free base (III) in 2-propanol. The resulting solution may be stored under inert conditions, for example under nitrogen. Preferably, the resulting solution of the free base (III) in 2-propanol is stored under nitrogen.

The solution of the racemic mixture of the free base (III) in 2-propanol may be resolved by introducing a resolving agent. The resolving agent may be, for example (1R, 3S)-(+)-camphoric acid, also known as D-camphoric acid.

Other resolving agents may include di-p-toluoyl-L-tartaric acid, L-tartaric acid, (1S)-(+)-10-camphorsulfonic acid or (1R)-(−)-10-camphorsulfonic acid. However, particularly preferred is D-camphoric acid.

A table showing contemplated solvents and conditions for reaction with D-camphoric acid as an exemplary resolving agent is shown below.

| Amount of | | | | |
|---|---|---|---|---|
| D-camphoric acid | | Solvents and | | |
| [in equivalents wrt 1 | | conditions with | Chiral | |
| mmol of (ii)] | Solvent | D-camphoric acid | Purity | Yield |
| 1 | 190 proof EtOH | RT then cool to −20° | — | |
| | (22.9 mL) | C. Seeded. Add | | |
| | | H₂O (20.6 mL), | | |
| | | seeded. | | |
| 1 | 190 proof EtOH | RT. | S/R: 98.9/1.1 | 15.1% |
| | (9.2 mL) | | | |
| 1 | IPA (9.2 mL) | RT. | S/R: 54/46 | 80.8% |
| 1 | EtOH (6.9 mL) | RT | S/R: 99.0/1.0 | 15.1% |
| | CH₃CN(6.9 mL) | | | |
| 1 | 190 proof EtOH | RT. | S/R: 97.0/3.0 | 33.4% |
| | (9.2 mL) and IPA | | | |
| | (9.2 mL) | | | |
| 1 | 190 proof EtOH | RT. | S/R: 95.8/4.2 | 41.8% |
| | (4.6 mL) and IPA | | | |
| | (9.2 mL) | | | |
| 1 | EtOH (6.0 mL) and | RT | S/R: 95.3/4.7 | 44.9% |
| | IPA (12.0 mL), | Recrystallisation | S/R: 99.8/0.2 | 71.5% |
| | | from EtOH (5.0 mL) | | |
| | | and IPA (10 mL) | | |
| 0.5 | EtOH (6.0 mL)and | RT 5 h. | S/R: 55/45 | 45.4% |
| | IPA (12.0 mL) | | | |
| 1.2 | EtOH (6.0 mL) and | RT 1 h. | S/R: 94.9/5.1 | 40.3% |
| | IPA (12.0 mL) | Recrystallization | S/R: 99.6/0.4 | 67.1% |
| | | from EtOH (5.0 mL) | | |
| | | and IPA | | |
| 1 | EtOH (6.0 mL) and | RT 2 h. | S/R: 94.3/5.7 | 45.8% |
| | IPA (12.0 mL) | Recrystallize from | S/R: 99.6/0.4 | 44.0% |
| | | IPA (30.0 mL). | | |
| | | Recrystallize from | S/R: 99.6/0.4 | 68.0 |
| | | IPA (40.0 mL). | | |
| | | Recrystallize from | S/R: 95.3/4.7 | 77.0% |
| | | EtOH (2.5 mL) and | | |
| | | IPA (5.0 mL). | | |
| | | Recrystallize from | S/R: 99.6/0.4 | 70.0% |
| | | EtOH (1.0 mL) and | | |
| | | IPA (2.0 mL), cool to | | |
| | | RT, add IPA | | |
| | | (10.0 mL). | | |
| 1 | EtOH (6.0 mL) and | RT 2 h. | S/R: 98.6/1.4 | 47.1% |
| | IPA(12.0 mL) | Reslurry in EtOH | S/R: 99.9/0.1 | 93.7% |
| | | (2.65 mL) and IPA | | |
| | | (3.31 mL) at 75-81° | | |
| | | C. for 1 h, cool to | | |
| | | 0-10° C., add IPA | | |
| | | (2.65 mL). | | |

With one equivalent of D-camphoric acid in 190 proof ethanol or a mixture of ethanol and water (approximate ratio of 1:1) no solids were observed, even after cooling to about −20° C. However, decreasing the volume of ethanol to approximately 10:1 with respect to the free base (III) solids were isolated with excellent chiral purity (S/R being 98.9/1.1).

Solids were also obtained using 2-propanol as a solvent. This solvent provided particularly high yield.

A mixture of ethanol and acetonitrile (in an amount of approximately 1:1) the chiral purity was achieved at approximately 99/1. The results were even more impressive for mixtures with ethanol and 2-propanol. Particularly preferred solvents for obtaining resolution were mixtures of ethanol and 2-propanol. Combinations of these mixtures provided superior resolution and yields.

The IPA:ethanol ratio may be between 1:4 and 4:1, preferably 1:1 and 1:3. A preferred ratio is 2:1.

The camphoric acid resolving agent is added to, e.g. dissolved in, an alcohol, for example ethanol, which is preferably absolute. The resulting reaction mixture, for example clear solution, may then be stirred under an inert atmosphere, for example under a nitrogen atmosphere, and may be heated to an internal temperature of 50 to 80° C., for example 60 to 70° C. (for example at an external temperature of 80 to 90° C.). The, e.g. solution, may be heated over a period of 20 to 40 minutes, for example 25 to 35 minutes, typically 30 minutes. To the resulting mixture may be added the racemic solution containing the free base (ii) in 2-propanol. The racemic mixture may be added to the e.g. solution containing the camphoric acid in ethanol, for example, over a period of 5 to 30 minutes, for example 10 to 20 minutes, typically 15 minutes. Typically, the internal temperature may be maintained at a temperature of 50 to 80° C., for example 60 to 70° C. during the addition of the racemic mixture. The resulting reaction mixture, for example a clear solution, may then be washed with additional alcohol, for example a propanol, typically 2-propanol. The exact volumes of each alcohol, for example the propanol and ethanol, more particularly the 2-propanol and the ethanol (absolute) are preferably established to achieve a v/v ratio of 2-propanol:ethanol at 2:1. In other words, the exact volumes of 2-propanol and ethanol in this step are important in preferred embodiments in order to achieve a v/v ratio of 2-propanol:ethanol as 2:1.

The resulting reaction mixture may then be seeded with the resolved camphoric acid salt, e.g. (S)-2-[2-(1-methyl-2-piperidinyl)ethyl]-benzenamine (1R,3S)-(+)-camphoric acid salt (iii). Following the addition of the resolved camphoric acid salt the reaction mixture may be cooled to a temperature of between 15 and 30° C., for example between 18 and 28° C., typically 23±3° C. The reaction mixture may be cooled over a period of between 1 and 2 hours.

However, it is noted that crystallization may occur at temperatures of about 55° C.

Then, the resulting reaction mixture may be stirred for an additional 2 hours at the aforementioned temperature. The resulting solid may be collected by filtration, e.g. over a polypropylene filter paper under suction. The solid may then be washed with an alcohol, for example a propanol, typically 2-propanol. Typically, the solid is washed with two equal portions of the aforementioned alcohol. The solid may then be dried, for example at a temperature of 40 to 55° C., typically 45 to 50° C. under a vacuum (13 to 40 mbar). The solid may be determined to be dry once the LOD is less than 1%. The resulting solid is that of crude (S)-2-[2-(1-methyl-2-piperidinyl)ethyl]-benzenamine (1R, 3S)-(+)-camphoric acid salt.

### Alternative Step B

(i) Free base generation:

A mixture, e.g. solution of 2-[2-(1-methyl-2-piperidinyl)ethyl]-benzenamine benzenesulfonate salt, for example a methylbenzenesulfonate salt, typically 4-methylbenzenesulfonate salt in an acetate, for example an alkyl acetate, typically isopropyl acetate or ethyl acetate may be stirred under an inert atmosphere, for example under a nitrogen atmosphere, at a temperature of 15 to 30° C., for example 20 to 28° C., typically 25° C. (internal temperature). Then, a solution of a base, e.g. an alkali metal hydroxide, particularly sodium hydroxide may be added. The sodium hydroxide solution may be added drop wise and this addition may be over a period of approximately 10 minutes, for example about 5 minutes. The reaction mixture may then be stirred for a further 5 to 60 minutes, for example 20 to 40 minutes, typically 15 to 30 minutes.

The solvent may also be any aromatic hydrocarbon, for example benzene, alkylbenzene, such as toluene or xylene or aromatic naphtha. In particular, toluene may be chosen as an alternative solvent.

The resulting reaction mixture may be phase-separated, for example, it may contain two or more separable phases, typically three separable phases such as an aqueous phase, an intermediate phase and an organic phase, for example. The aqueous phase may be removed. The remaining phase or phases may then be passed over a filter. Then, an acetate, for example an alkyl acetate, typically isopropyl acetate may be added. The acetate may be added portion-wise. Then the reaction mixture may be reduced in volume, for example under vacuum (150 to 250 mbar typically 220 mbar) and at a temperature of 40 to 65° C., for example 50 to 60° C., typically 55° C. Then, the reduced-volume reaction mixture may be further treated with an acetate, for example an alkyl acetate, typically isopropyl acetate. Preferably, the resulting reaction mixture, i.e. the product in acetate, is water-free.

### (ii) Addition of Resolving Agent:

The organic phase, for example water-free organic phase, may then be subjected to an elevated temperature, for example a temperature of between 55 and 100° C., for example 70 and 90° C., typically 85° C. The reaction mixture may be stirred. Then, a mixture, for example solution, of a resolving agent, for example camphoric acid in an alcohol, for example isopropanol, may be added to the reaction mixture. The addition of the resolving agent may be conducted over a period of approximately 10 minutes, for example about 5 minutes. The resulting reaction mixture, for example solution, may then be treated with a further addition of an alcohol, for example isopropanol. The resulting reaction mixture may then be stirred at an external temperature of 50 to 100° C., for example 70 to 90° C., typically 85° C. The reaction mixture may be stirred for approximately 30 minutes. Then, the reaction mixture may be cooled to an internal temperature of approximately 50 to 70° C., typically 60° C. and treated with a mixture, for example, a suspension, of the resolved salt (iiiA) in an acetate, for example an alkyl acetate, typically isopropyl acetate. Then, the reaction mixture may be allowed to cool to approximately room temperature (for example 20 to 28° C., typically 22 to 27° C.) over a period of approximately 1 hour and may then be stirred at room temperature for a further period of time, for example a further hour. Then, a mixture of an acetate (for example an acetic acid ester) and an alcohol may be added to the reaction mixture. The acetate may be, for example, an alkyl acetate, typically isopropyl acetate and the alcohol may isopropanol. The mixture of the alcohol/acetate may be in a ratio of acetate:alcohol of 4:1 g/g. The resulting product may then be dried under a vacuum at a temperature of approximately 40 to 60° C., for example 55° C.

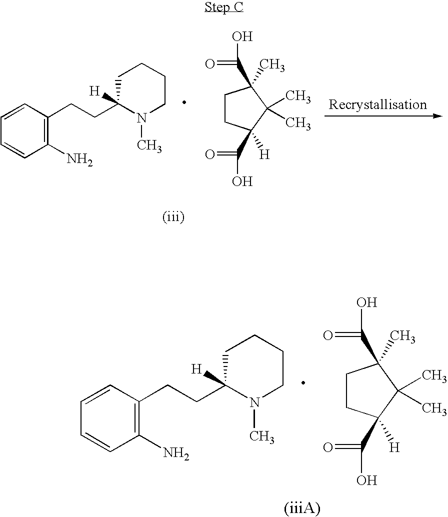

The crude camphoric acid salt (iii) may be treated with an alcohol, for example ethanol, typically absolute ethanol in admixture with a second alcohol, for example a propanol, typically 2-propanol. The resulting reaction mixture may then be agitated at a temperature of 15 to 30° C., for example 18 to 28° C., typically 23±3° C. The resulting reaction mixture, for example thick slurry, may then be heated to an internal temperature of 70 to 85° C., typically 78±3° C. (external temperature 85 to 95° C.). The reaction may be heated over a period of approximately 1 hour. The reaction mixture may undergo gentle refluxing. The resulting reaction mixture, for example light suspension, may then be stirred at a temperature of 78±3° C. for an additional hour. The reaction mixture may then be cooled to a temperature of 23±3° C. over a period of 1 to 2 hours. The resulting reaction mixture, for example thick slurry, may then be agitated, e.g. stirred and cooled to a temperature of 5±5° C. (external temperature 0 to 5° C.). The reaction mixture may be cooled over a period of approximately 30 minutes. Then, an alcohol, e.g. a propanol, typically 2-propanol may then be added to the reaction mixture. The resulting diluted reaction mixture may then be stirred. The resulting mixture e.g. suspension, may then be stirred for an additional 30 minutes, for example at a temperature of 5±5° C. The resulting solid may then be collected by filtration e.g. over a polypropylene filter paper under suction. The solid may then be washed with an alcohol e.g. a propanol, typically 2-propanol. The wash may be carried out in 2 equal portions. The solid may then be dried at a temperature of 40 to 55° C., typically 45 to 50° C. under vacuum conditions (13 to 40 mbar). The resulting product is considered to be dry when the LOD is less than 1%.

### Alternative Step C

A mixture, for example a solution, of the crude camphoric acid salt (iii) in ethanol, for example absolute ethanol, may be treated with an alcohol, for example isopropanol (2-proponol). The reaction mixture may then be heated to an elevated temperature, for example to a reflux temperature, such as 70 to 100° C. (external temperature) typically 90° C. (external temperature) which may provide an internal temperature of approximately 75° C. The reaction mixture may be refluxed for a period of about 20 to 40 minutes, typically 30 minutes, at an internal temperature of 55 to 85° C., for example 65 to 75° C., typically 75° C. The reaction mixture may be stirred. The reaction mixture may then be cooled, for example over a period of 2 hours, typically over a period of 2 hours, to an internal temperature of 10 to −10° C., typically 0° C. The resulting reaction mixture, for example suspension, may then be heated to an elevated temperature, for example a temperature of approximately 65° C., typically 65° C. The reaction mixture, for example suspension, may be heated in an ultrasonic bath. The reaction mixture may then be seeded with the resolved camphoric acid salt (iiiA) in an alcohol, for example isopropanol. Then, the reaction mixture may be cooled to an internal temperature of between −10 and 15° C., for example 0 to 5° C., over a period of, for example, about 15 minutes. Then, an alcohol, for example isopropanol, may be added. The alcohol may be added over a period of approximately 30 minutes. The reaction mixture may be stirred. The temperature may be maintained at an internal temperature of between −10 and 15° C., typically 0 to 5° C. Then, at an internal temperature of −10 to 15° C., further alcohol, for example isopropanol, may be added. The resulting reaction mixture may then be filtered. Preferably, the reaction mixture is maintained at a temperature of −10 to 15° C., for example 0 to 5° C., typically 0° C. during filtration. The resulting solid may then be dried, for example under vacuum conditions, and at a temperature of 40 to 60° C., for example 55° C.

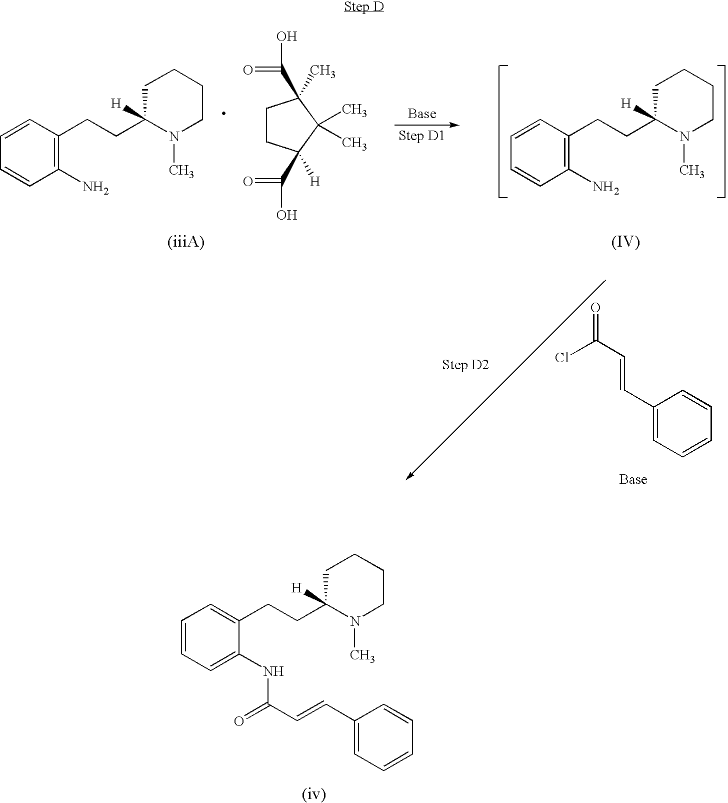

### Step D1

The resolved camphoric acid salt may be treated with an acetate, for example an alkyl acetate, typically isopropyl acetate or ethyl acetate. The resulting reaction mixture may then be stirred at a temperature of 15 to 30° C., for example 20 to 25° C. The reaction mixture may be under an inert atmosphere, for example a nitrogen atmosphere. Then, a solution of a base, e.g. sodium hydroxide, in water may be added to the reaction mixture over a period of approximately 5 minutes whilst maintaining an internal temperature of 15 to 30° C.

The solvent may also be any aromatic hydrocarbon, for example benzene, alkylbenzene, such as toluene or xylene or aromatic naphtha. In particular, toluene may be chosen as an alternative solvent.

The resulting reaction mixture, for example suspension, may then be stirred until all of the solid dissolves, for example about 5 minutes. The resulting two-phase reaction mixture may then be separated and the aqueous layer may be washed with an acetate, for example an alkyl acetate, typically isopropyl acetate. The organic layers may then be combined and may be washed with water. The organic layer may then be concentrated under a vacuum (for example 10 to 100 mbar, typically 20 to 100 mbar) at an internal temperature of approximately 20 to 40° C. (external temperature 30 to 60° C.). The resulting reduced-volume solution may then be stored under inert conditions, for example under nitrogen conditions. The resulting product is a solution of the free base (S)-2-[2-(1-methyl-2-piperidinyl)ethyl]-benzenamine (±2) in isopropyl acetate.

### Step D2

The aforementioned mixture, e.g. solution of the free base in, e.g. isopropyl acetate, may then be treated with a carbonate, for example potassium carbonate. The reaction mixture may then be stirred under inert conditions, for example nitrogen conditions at an internal temperature of 15 to 30° C., for example 23±3° C. The resulting reaction mixture, for example suspension, may then be treated with cinnamoyl chloride over a period of approximately 5 minutes, whilst maintaining an internal temperature of 15 to 30° C. The resulting reaction mixture, for example slurry, may then be heated to an elevated temperature, for example to an internal temperature of between 70 and 100° C., for example 85±5° C. (external temperature 90 to 100° C.) over a period of 30 to 60 minutes. The reaction mixture may then be stirred at the aforementioned temperature for an additional 2 hours. The reaction mixture may then be cooled to a temperature of 15 to 30° C. (for example 23±3° C. over a period of 1 hour. The resulting, cooled, reaction mixture may then be treated with water. The resulting reaction mixture may then be stirred at a temperature of 15 to 30° C., for example 23±3° C. for a period of 30 to 60 minutes, for example, to obtain a two-phase solution. The layers may be separated.

To the organic layer, may be added a solution of inorganic acid, for example HCl, typically 0.5 N HCl. The HCl solution may be added over a period of approximately 10 minutes, whilst maintaining an internal temperature of 15 to 30° C. The resulting bi-phase solution was then further separated. To the aqueous layer may be added an acetate, for example an alkyl acetate, typically isopropyl acetate. The reaction mixture may then be stirred and a solution of a base, for example sodium hydroxide, in water may then be added over a period of approximately 10 minutes whilst maintaining an internal temperature of 15 to 30° C. The resulting two-phase solution may then be separated and the organic layer saved. The aqueous layer may then be extracted by an acetate, for example an alkyl acetate, typically isopropyl acetate or ethyl acetate. The organic layers may then be combined and may be washed with water. The organic layer may then be concentrated under vacuum (for example 10 to 110 mbar, typically 20 to 100 mbar) at an internal temperature of 20 to 40° C. (external temperature 30 to 60° C.). The resulting product is a solution of (iv) in isopropyl acetate.

The solvent may also be any aromatic hydrocarbon, for example benzene, alkylbenzene, such as toluene or xylene or aromatic naphtha. In particular, toluene may be chosen as an alternative solvent.

The product may then be stirred and heated to an internal temperature of 70 to 100° C. (for example 85±5° C. (external temperature 90 to 100° C.)) over a period of 30 to 60 minutes. Then, the reaction mixture may be treated with heptane over a period of approximately 10 minutes, whilst maintaining an internal temperature of 70 to 100° C. The resulting reaction mixture may then be stirred and cooled to a temperature of 15 to 30° C., for example 23±3° C. over a period of approximately 1 hour. It is noted that crystallization may occur at a temperature of 45 to 55° C. The resulting reaction mixture, for example slurry, may then be stirred for a further 2 hours at a temperature of 15 to 30° C. The resulting solid may then be collected by filtration, for example over a polypropylene filter paper under suction. The resulting solid may then be washed with a mixture of an acetate and an alkane, for example a mixture of an alkyl acetate and an alkane, typically a mixture of isopropyl acetate and heptane. The ratio of isopropyl acetate to heptane may be for example in the region of 1:6. The solid may be washed in two equal portions. The solid may then be dried at a temperature of 40 to 55° C., for example 45 to 50° C. under a vacuum (typically 13 to 40 mbar). The solid may be determined to be dry when the LOD is less than 1%. The resulting product, (2E)-N-[2-[2-[(2S)-1-Methyl-2-piperidinyl]ethyl]phenyl]-3-phenyl-2-propenamide is therefore isolated.

The present invention provides a reaction step using an acetate, typically isopropyl acetate in a base, typically sodium hydroxide. This process has much greater environmental benefits than the prior art process, which used dichloromethane at this stage in the synthesis of compound (I).

The isopropyl acetate solution, for example, is concentrated to azeotropically remove water and to obtain a dry free base solution. The presence of a dry solution means that the reaction mixture may be used directly without further purification.

The coupling of the free base (I2) with cinnamoyl chloride may also be carried out in an acetate, typically isopropyl acetate, in the presence of potassium carbonate.

A possible reaction by-product may be present as an impurity of formula (II):

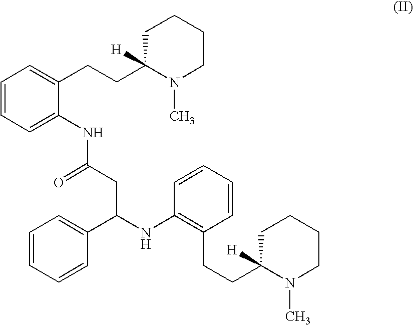

Below is a table giving further contemplated reaction conditions for the coupling of the free base (IV) of the compound (iiiA) with cinnamoyl chloride. It will of course be understood that the conditions disclosed herein are examples and are not intended to be limiting:

| Reaction Conditions | Results | Observatons |
|---|---|---|
| To (IV) free base (1 eq) and K₂CO₃ (3 | (II) not detected | (IV) free base solution |
| eq) in IPAc at RT, add Cin-Cl | (iv) Yield: 78% | was concentrated to |
| (cinnamoyl chloride) (1.5 eq) in IPAc in 5 | Purity: 99.8% | dryness. |
| min, warm to reflux, stir 2 h, cool to RT, | | |
| add water; | | |
| Separate organic layer, 0.5 N HCl then 2 | | |
| N NaOH workup, (iv) crystallize from | | |
| Heptane/IPAc (6/1) | | |
| To (IV) free base (1 eq) in IPAc at RT, | (IV)/(iv) = 3.1% | (IV) free base solution |
| add Cin-Cl (1.5 eq) in IPAc in 1~2 min, | (II)/(iv) = 11.0% | was not concentrated. |
| stir 30 min, add K₂CO₃ in water at RT | | |
| To (IV) free base (1 eq) and K₂CO₃ (2 | 1.5 eq Cin-Cl: | (IV) free base solution |
| eq) in IPAc and water at RT, add Cin-Cl | (IV)/(iv) = 1.2%; | was not concentrated; |
| (1.5 eq) in IPAc in 20 min, stir 1 h; | +0.1 eq Cin-Cl: | reaction mixture was a |
| then add Cin-Cl (0.1 eq) in IPAc | (IV)/(iv) = 0.7% | bi-phasic solution; (II) |
| | (II)/(iv) = 42.2% | was 1.2% after |
| | | crystallization from |
| | | Hep/IPAc (4/1). |
| To (IV) free base (1 eq) and NaHCO₃ (5 | (IV): n.d. | (IV) free base solution |
| eq) in IPAc and water at 0~5° C., add | (II)/(iv) = 33% | was not concentrated; |
| Cin-Cl (1.5 eq) in IPAc in 30 min, warm | | reaction mixture was a |
| to RT, stir 30 min | | bi-phasic solution. |
| To (IV) free base (1 eq) and 4- | (IV)/(iv) = 0.7% | (IV) free base solution |
| methylmorpholine (2 eq) in IPAc at RT, | (II)/(iv) = 4.3% | was not concentrated. |
| add Cin-Cl (1.5 eq) in IPAc in 5~10 min, | | |
| stir 30 min | | |
| To (IV) free base (1 eq) in CH₂Cl₂ at 0~5° | (IV): n.d. | reaction mixture was a |
| C., add Cin-Cl (1.1 eq) in CH₂Cl₂ in 2~3 | (II)/(iv) = 69.6% | clear solution. |
| min, warm to RT, stir 30 min | | |
| To (IV) free base (1 eq) and K₂CO₃ (3 | (IV): n.d. | (IV) free base solution |
| eq) in NMP at RT, add Cin-Cl (1.5 eq) in | (I)/(iv) = 1.7% | was concentrated to |
| NMP in 5~10 min, stir 30 min; | | dryness; reaction |
| Add water to the reaction mixture: oil out | | mixture was a clear |
| | | solution except K₂CO₃. |
| To (IV) free base (1 eq) and K₂CO₃ (3 | (IV): n.d. | (IV) free base solution |
| eq) in NMP at RT, add Cin-Cl (1.3 eq) in | (I)/(iv) = 1.3% | was concentrated to |
| NMP in 5~10 min, stir 30 min; | | dryness; reaction |
| Add everything into water: became a | | mixture was a clear |
| hard chuck when K₂CO₃ was added. | | solution except K₂CO₃. |
| To (IV) free base (1 eq) in NMP at 0~10° | (IV): n.d. | (IV) free base solution |
| C., add Cin-Cl (1.1 eq) in NMP in 5~10 | (II)/(iv) = 7.4% | was concentrated to |
| min, warm to RT, stir 30 min; | | dryness; reaction |
| Add into 0.2N NaOH: white solid formed | | mixture was a clear |
| first, became gummy solid later | | solution. |
| To Cin-Cl (1.5 eq) in IPAc at RT, add | (IV)/(iv) = 5.4% | (IV) free base solution |
| (IV) free base (1 eq) in IPAc in 10 min, | (II): n.d. | was concentrated to |
| stir 30 min, filter. | | dryness; good solid |
| | | formed. |
| To Cin-Cl (1.5 eq) in IPAc at RT, add | (IV)/(iv) = 3.1% | (IV) free base solution |
| (IV) free base (1 eq) in IPAc in 15 min, | (II)/(iv) = 0.4% | was concentrated to |
| stir 30 min, filter, dry; | (iv) Yield: 73% | dryness; good solid |
| Dissolve in IPAc and 1 N NaOH, (iv) | Purity: 99.8% | formed; no (II) and (IV) |
| crystallize from Heptane/IPAc (5/1) | | detected after |
| | | crystallization. |
| To Cin-Cl (1.5 eq) in IPAc at RT, add | (IV)/(iv) = 0.6% | (IV) free base solution |
| (IV) free base (1 eq) in IPAc in 5~10 | (II)/(iv) = 0.5% | was not concentrated. |
| min, stir 30 min. | | |
| To Cin-Cl (1.5 eq) in Toluene at RT, | (IV)/(iv) = 0.3% | (IV) free base solution |
| add (IV) free base (1 eq) in Toluene in | (II)/(iv) = 0.3% | was not concentrated. |
| 5~10 min, stir 30 min | | |

where IPAc is isopropyl acetate and Cin-Cl is cinnamoyl chloride. It is of course contemplated that other solvents as herein described as alternatives may be used. As such, ethyl acetate is an example.

The resulting product is preferably of high in chiral purity, for example over 95% pure, typically over 99% pure. In a particular embodiment of the present invention no R-enantiomer is detectable by chiral HPLC.

The conditions and procedure of the present invention provide that the impurity (II) is formed in an amount of less than 5%, for example less than 3%, typically less than 1%, measured by HPLC. The conditions of the present invention that allow this reduction in by-products are the addition of a solution of the free base (I2) in isopropyl acetate into the cinnamoyl chloride solution in isopropyl acetate at room temperature. The reaction is very fast and the acid salt of the compound (iv) precipitate out immediately as a white solid.

### Alternative Step D1

A mixture, for example solution of the resolved camphoric acid salt (iiiA) in an acetate, for example an alkyl acetate, typically isopropyl acetate may be subjected to a temperature of 15 to 30° C., for example 20 to 25° C. The reaction mixture may be stirred. Then, at an internal temperature of 20 to 35° C., for example 25 to 30° C. (typically an external temperature of 20° C., a solution of a base, for example sodium hydroxide, may be added. The addition of the base may be conducted over a period of approximately 5 minutes. The resulting reaction mixture, for example, suspension, may then be stirred for a period of 15 to 45 minutes, typically 30 minutes. The resulting reaction mixture, for example emulsion, typically an orange emulsion, may then be allowed to separate into a two-phase mixture. The water phase may be removed. The remaining organic phase may then be reduced in volume, for example by use of a rotary evaporator, and the acetate may be distilled off at a temperature of 50 to 70° C., typically 60° C. and under reduced pressure (220 to 260 mbar, typically 250 mbar).

### Alternative Step D2.

The aforementioned reaction mixture may then be treated with a ketone, for example 2-butonone and an acetate, for example an alkyl acetate, typically isopropyl acetate at a temperature of 25 to 40° C., typically about 35° C. (for example external temperature 38° C.). The reaction mixture may be under inert conditions, for example under nitrogen. Then, at an internal temperature of approximately of approximately 35° C., a solution of cinnamoyl chloride in 2-butonone, for example may be added. The solution containing cinnamoyl chloride may be added drop wise. Then, further ketone solvent may be added. The resulting reaction mixture, for example suspension, may then be stirred for a period of approximately 15 to 30 minutes, typically 20 minutes, at an internal temperature of about 35° C. The pH of the reaction mixture may be between 5 and 9, for example between 6 and 8, typically 7.

The resulting reaction mixture, for example suspension, may then be cooled to an internal temperature of 20 to 30° C., typically 25° C. and water and an acetate, for example an alkyl acetate, typically isopropyl acetate may be added. The resulting reaction mixture may then be stirred for a period of 5 to 25 minutes, typically about 15 minutes at an internal temperature of 20 to 30° C., typically 25° C. (for example external temperature of 20° C.). The resulting reaction mixture, for example two-phase reaction mixture, may then be separated. The water phase may be removed. The upper layer, typically yellow in colour, may then be treated with an acid, for example hydrochloric acid. The resulting two-phase reaction mixture may then be separated. The remaining organic phase may then be washed with further acid and the resulting two-phase mixture may be further separated and the water phase combined with the first water phase. The combined water phases may then be treated with acetic acid and a base, for example sodium hydroxide. The resulting reaction mixture may then be stirred at an internal temperature of 20 to 35° C., typically 25 to 30° C. (for example, an external temperature of 20° C.). The reaction mixture may be stirred for a period of 10 to 30 minutes, for example 10 to 20 minutes, typically 15 minutes. The resulting two-phase reaction mixture may then be separated and the aqueous phase discarded.

The organic phase may then be reduced in volume, for example on a rotary evaporator and at an external temperature of 50 to 70° C., typically 60° C. and under vacuum, for example 220 to 260 mbar. Then, the reduced-volume reaction mixture may be treated with an alcohol, for example isopropanol. The resulting reaction mixture may then be reduced in volume, for example on a rotary evaporator, at an external temperature of 50 to 70° C., typically 60° C. and under a vacuum, for example 120 to 180 mbar, typically 150 mbar. Then, the reaction mixture may be treated with water at an internal temperature of 45 to 60° C., for example 50 to 55° C. (typically an external temperature of about 60° C.). The resulting reaction mixture for example, suspension, may then be further treated with product (iv) in an alcohol, for example isopropanol. The reaction mixture may then be stirred for a period of 5 to 30 minutes, for example 10 to 20 minutes, typically 15 minutes. The reaction mixture may be stirred at an internal temperature of 45 to 60° C., for example 50 to 55° C. Then, the reaction mixture may be treated with further water which may be added over a period of 5 to 45 minutes, for example 15 to 30 minutes. The reaction mixture may be maintained at an internal temperature of 45 to 60° C. Then, the resulting reaction mixture, for example suspension, may be cooled to an internal temperature of 15 to 30° C., for example 20 to 22° C. Then, the resulting reaction mixture, for example suspension, may be stirred for a period of 15 to 45 minutes, typically about 30 minutes and at an internal temperature of 15 to 30° C., for example 20 to 25° C., typically 20 to 22° C. The resulting reaction mixture may then be filtered and the solid collected. The solid may be washed with a mixture of water and an acetate, where the water:acetate ratio is approximate 5:1 g/g. The acetate may be, for example, an alkyl acetate, typically isopropyl acetate or ethyl acetate. The solid may then be dried under a vacuum and at a temperature of 40 to 60° C., for example 45 to 55° C., typically 55° C.

The solvent may also be any aromatic hydrocarbon, for example benzene, alkylbenzene, such as toluene or xylene or aromatic naphtha. In particular, toluene may be chosen as an alternative solvent.

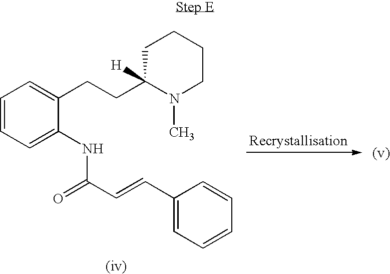

The product (iv) may be added to an alcohol, for example isopropanol and an alkane, for example a heptane fraction from petroleum having a boiling point of 65 to 100° C. Then, the reaction mixture may be heated to an internal temperature of 50 to 85° C., for example about 75° C. (typically an external temperature of 95° C.). The reaction mixture may be refluxed. The reaction mixture may be heated for a period of between 15 and 45 minutes, typically 30 minutes. The reaction mixture may be stirred. Then, the reaction mixture may be filtered. The reaction mixture may be at an internal temperature of 60 to 80° C., for example 70 to 75° C., typically an external temperature of 85° C. The resulting reaction mixture may then be treated with a mixture of an alcohol and an alkane, for example isopropanol and heptane. The resulting reaction mixture may then be heated to an internal temperature of approximately 70° C., typically an external temperature of 95° C. The reaction mixture may be stirred. Then, further heptane may be added. The heptane may be added drop wise. The reaction mixture may be maintained at an internal temperature of 50 to 80° C., for example 65 to 75° C., typically an external temperature of 75° C.

The resulting solution may then be cooled to an internal temperature of 30 to 50° C., typically 40° C. (for example an external temperature of 40° C.). The reaction mixture may be cooled over a period of 5 to 30 minutes, for example 10 to 20 minutes, typically 15 minutes. Then, at an internal temperature of 30 to 50° C., typically 40° C., the resulting reaction mixture, for example solution, may be treated with a mixture, for example suspension, of the product (v) in an alkane, for example heptane. The resulting reaction mixture may then be stirred for a period of 15 to 45 minutes, for example 20 to 35 minutes, typically 30 minutes, at an internal temperature of 30 to 50° C., typically 40° C. Then, the resulting reaction mixture may be treated with further alkane, for example heptane. The resulting reaction mixture, for example suspension, may then be cooled to an internal temperature of −15 to 0° C., for example −10 to −5° C., typically −10° C. (external temperature of, for example, −10 to −15° C.). The reaction mixture may be cooled over a period of 15 to 45 minutes, typically about 30 minutes. The reaction mixture may be stirred for a further 40 to 90 minutes, for example about 60 minutes. The resulting reaction mixture may then be filtered at an internal temperature of approximately −10° C. The resulting solid may then be washed in a mixture of an alcohol and an alkane, for example a mixture of isopropanol and heptane. The mixture of the alcohol and alkane may be in a ratio of alcohol:alkane of 1:1.5. The solid may then be washed with the aforementioned mixture. The solid may then be dried for example under a vacuum, at a temperature of approximately 50 to 70° C., typically about 60° C.

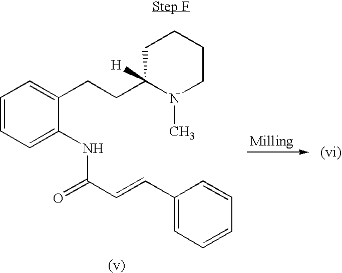

The product (v) may be further processed, for example by milling, to produce a fine particle product.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The invention will now be further exemplified by the non-limiting examples below:

### EXAMPLES

### Example 1

### Synthesis of 2-[2-(1-Methyl-2-piperidinyl)ethyl]-benzenamine 4-methylbenzenesulfonate

An MP-10 vessel is put under an inert atmosphere by pressurizing with nitrogen to e.g. 4.5 bar, then depressurizing to 1 bar. Repeat this pressurization/depressurization four times. Charge the MP-10 vessel with 43.90 g of 1-methyl-2-[(E)-2-(2-nitrophenyl)-ethenyl]-pyridinium 4-ethylbenzenesulfonate. Inert the vessel with nitrogen as described above. Add 1.87 g of 10% Pt/C (62.4% wet). Inert the vessel with nitrogen as described above. Add 395.6 g of methanol. Inert the vessel with nitrogen as described above. Stir the vessel at 450 rpm, set the batch temperature at 30° C., and allow the batch temperature to equilibrate at 30° C. Set the temperature control of the RC1 to Tj mode and turn off the agitator. Purge the headspace of N₂, and replace with H₂ by pressurizing with H₂ to 4.5 bar, depressurizing to 1 bar. Repeat the H₂ pressurization/depressurization cycle 4 times. After the final depressurization, set the reactor pressure to 5.2 bar, agitate at 450 rpm to start the reaction, and switch the RC1 to Tr mode. The initial reaction is exothermic, giving a maximum heat evolution rate of about 35 W/kg (excepting for a short-lived spike with a maximum of ˜50 W/kg). Reaction start is detected immediately, based on hydrogen uptake and heat evolution. Hydrogenate at 30° C. and 5.2 bar for 7.2 h. Depressurize the reactor to 1 bar, and purge with N₂ by pressurizing to 4.5 bar and depressurizing as described above (5 cycles). Empty the reactor, and rinse the MP-10 vessel with: 44.8 g of methanol and combine the rinse with the reaction mixture. Filter the batch over a pad of 8.0 g of Celite. Wash the Celite pad with 39.6 of methanol and combine the filtrate [caution: do not allow the cake to dry; solid catalyst is flammable]. Charge the filtrate into a 1-L LabMax, distill the filtrate at an internal temperature at 35-45° C. (jacket temperature: 65-75° C.) under reduced pressure (80-160 mbar) to collect 450 mL of solvent (batch volume: ˜150 mL). Add to the batch 353.3 g of peroxide-free 2-propanol. Distill the batch at an internal temperature at 35-45° C. (jacket temperature: 65-75° C.) under reduced pressure (80-160 mbar) to collect 450 mL of solvent (batch volume: ˜150 mL). Add 353.3 g of 2-propanol. Distill the batch at an internal temperature at 35-45° C. (jacket temperature: 65-75° C.) under reduced pressure (80-160 mbar) to collect 450 mL of solvent (batch volume: ˜150 mL). Heat the batch to an internal temperature at 60±5° C. over a period of 20 min and add 43.7 g of isopropyl acetate over a period of 20 min while maintaining the internal temperature at 55-65° C. Cool the mixture to an internal temperature at 40±5° C. over a period of 20 min and seed the batch with 160 mg of pure A6. Cool the suspension to an internal temperature at 20±5° C. over a period of 1 h and stir at this temperature for an additional 4 h. Collect the solid by filtration over a Büchner funnel with suction, wash the solid with 2×42.1 g of 2-propanol/isopropyl acetate (1:2 v/v). Dry the solid under reduced pressure (15-40 mbar) at 60° C. until an LOD of <1% is reached to afford 26.3 g of 2-[2-(1-methyl-2-piperidinyl)ethyl]benzenamine 4-methylbenzenesulfonate (1:1).

Theoretical Yield: 41.60 g

Yield: 63.2%

Purity: 98.8%

Melting Point: 133-135° C.

### Example 2

### Synthesis of (S)-2-[2-(1-Methyl-2-piperidinyl)ethyl]-benzenamine (1R,3S)-(+)-camphoric acid salt (1:1)

### (a) Free Base Generation

A 500-mL, 4-necked, round-bottomed flask, equipped with a mechanical stirrer, digital thermometer and nitrogen inlet-outlet, heating cooling bath, and addition funnel, is charged with 30.00 g of 2-[2-(1-methyl-2-piperidinyl)ethyl]-benzenamine 4-methylbenzenesulfonate salt (1:1) and 200 mL of isopropyl acetate. Stir the mixture at 20-25° C. under nitrogen and add a solution of 4.00 g of sodium hydroxide in 50 mL of water over a period of 5 min while maintaining an internal temperature at 20-25° C. Stir the suspension efficiently until all the solid dissolved (5 min). Separate the organic layer and save. Extract the aqueous layer with 67 mL of isopropyl acetate. Combine the organic layers and wash it with 50 mL of water. Separate the organic layer and concentrate it under vacuum (20-100 mbar) at an internal temperature at 20-40° C. (external temperature 30-60° C.) to obtain 20˜30 mL of a solution. Add 50 mL of 2-propanol and concentrate it under vacuum (20-100 mbar) at an internal temperature at 20-40° C. (external temperature 30-60° C.) to obtain 20˜30 mL of a solution. Add 50 mL of 2-propanol and concentrate it under vacuum (20-100 mbar) at an internal temperature at 20-40° C. (external temperature 30-60° C.) to obtain 20˜30 mL of a solution. Add ˜100 mL of 2-propanol to obtain 95.27 g (117 mL) of a solution of (±)-2-[2-(1-methyl-2-piperidinyl)ethyl]-benzenamine (containing 16.77 g of free base) in 2-propanol. Save this solution for the next step and store it under nitrogen.

### (b) Resolution

A 500-mL, 4-necked, round-bottomed flask, equipped with a mechanical stirrer, digital thermometer, nitrogen inlet-outlet, heating mantle, condenser, and addition funnel is charged with 15.38 g of (1R,3S)-(+)-camphoric acid and 100 mL of 200 proof ethanol to afford a clear solution. Stir the solution under nitrogen and heat to an internal temperature at 65±5° C. (external temperature 80-90° C.) over a period of 30 min. Add a solution of 95.27 g (117 mL) of a solution of (±)-2-[2-(1-methyl-2-piperidinyl)ethyl]-benzenamine (containing 16.77 g of free base) in 2-propanol over a period of 15 min while maintaining an internal temperature at 65±5° C. to obtain a clear solution. Wash the addition funnel with 100 mL of 2-propanol and add to the reaction mixture. Add 10 mg of (S)-2-[2-(1-methyl-2-piperidinyl)ethyl]-benzenamine (1R,3S)-(+)-camphoric acid salt (1:1) seeds and cool the reaction mixture to 23±3° C. over a period of 1˜2 h. Stir the mixture at 23±3° C. for an additional-2 h. Collect the solid by filtration over a polypropylene filter paper in a Buchner funnel with suction. Wash the solid with a total of 100 mL of 2-propanol in two equal portions of 50 mL each. Dry the solid at 45-50° C. under vacuum (13-40 mbar) with nitrogen bleeding to obtain a constant weight (LOD<1%, 4 h) of 15.14 g of crude (S)-2-[2-(1-methyl-2-piperidinyl)ethyl]-benzenamine (1R,3S)-(+)-camphoric acid salt (1:1) as a white solid (enantiomeric purity: S:R=98.6:1.4).

### (c) Enrichment of crude (S)-2-[2-(1-methyl-2-piperidinyl)ethyl]-benzenamine (1R,3S)-(+)-camphoric acid salt (1:1)

A 250-mL, 4-necked, round-bottomed flask, equipped with a mechanical stirrer, digital thermometer, nitrogen inlet-outlet, heating mantle, condenser, and addition funnel is charged with 15.10 g of crude (S)-2-[2-(1-methyl-2-piperidinyl)ethyl]-benzenamine (1R,3S)-(+)-camphoric acid salt, 40 mL of 200 proof ethanol, and 50 mL of 2-propanol. Agitate the reaction mixture at 23±3° C. to obtain a thick slurry. Heat the mixture to an internal temperature at 78±3° C. (external temperature 85-95° C.) over a period of 1 h to achieve a gentle refluxing. Stir the light suspension at 78±3° C. for an additional 1 h. Cool the reaction mixture to 23 ±3° C. over a period of 1˜2 h. Agitate the resulting thick slurry and cool to 5±5° C. (external temperature 0-5° C.) over a period of 30 min. Add 40 mL of 2-propanol to dilute the reaction mixture and agitate the resulting suspension at 5±5° C. for an additional 30 min. Collect the solid by filtration over a polypropylene filter paper in a Buchner funnel with suction. Wash the solid with a total of 60 mL of 2-propanol in two equal portions of 30 mL each. Dry the solid at 45-50° C. (13-40 mbar) with nitrogen bleeding to obtain a constant weight (LOD<1%, 4 h) of 14.16 g of (S)-2-[2-(1-methyl-2-piperidinyl)ethyl]-benzenamine (1R,3S)-(+)-camphoric acid salt (1:1) as a white solid.

Theoretical Yield: 32.15 g

Yield: 44.0%

Enantiomeric Purity: S:R=99.9:0.1 (Chiral HPLC area %).

### Example 3

### Alternative synthesis of (S)-2-[2-(1-Methyl-2-piperidinyl)ethyl]-benzenamine (1R,3S)-(+)-camphoric acid salt-(1:1)

### (a) Synthesis of (S)-2-[2-(1-Methyl-2-piperidinyl)ethyl]-benzenamine (1R,3S)-(+)-camphoric acid salt

### (i) Free Base Generation

In a 250 ml round bottomed flask equipped with a magnetic stirrer 2-[2-(1-Methyl-2-piperidinyl)ethyl]-benzenamine 4-methylbenzinesulphonate (10 g) and isopropyl acetate (60 ml) were added under a nitrogen atmosphere and stirred at an internal temperature of 25° C. Then, at 25° C. a solution of sodium hydroxide (4.23 g) in water (30 ml) is added drop wise to the reaction mixture over a period of 5 minutes. Then the reaction mixture is stirred for a further 15 to 30 minutes.

In a separating funnel, the clear bright yellow water phase is separated from the intermediate phase. The intermediate phase and the organic phase may then be passed over a glass fibre filter into a 250 ml round bottom flask. The water phase is approximately 40 ml and the combined intermediate and organic phases is approximately 80 ml. Then, to the filtered mixture is added isopropyl acetate (10 ml) portion wise. Then, the reaction mixture is reduced in volume at a temperature of 55° C. and under reduced pressure of 220 mbar. Then, isopropyl acetate is added to the reaction mixture in 2-portions (2×65 ml).

### (ii) Synthesis of (S)-2-[2-(1-Methyl-2-piperidinyl)ethyl]-benzenamine (1R,3S)-(+)-camphoric acid salt

The water-free organic phase is subjected to an external temperature of 85° C. and stirred. Then, a solution of camphoric acid (5.44 g) in isopropanol (11.5 ml) is added over a period of 5 minutes and then the solution is treated with a further addition of isopropanol (2×3 ml). The reaction mixture is then stirred at an external temperature of 85° C. for 30 minutes. Then, the reaction mixture is taken to an internal temperature of 60° C. and treated with a suspension of the resolved camphoric acid salt (IV) (10 mg) and isopropyl acetate (0.15 ml) is further added. Then, the reaction mixture is allowed to cool to room temperature over a period of approximately one hour and is then stirred at room temperature for a further hour. Then, a mixture of isopropyl acetate and isopropanol in a ratio of isopropyl acetate:isopropanol of 4:1 g/g is added to the reaction mixture. The resulting product is then dried under vacuum at a temperature of 55° C.

Yield: 4.35 g (40.6% of theory)

### (b) Recrystallisation of (S)-2-[2-(1-Methyl-2-piperidinyl)ethyl]-benzenamine (1R,3S)-(+)-camphoric acid salt

In a 100 ml vessel with a mechanical stirrer and under an inert (nitrogen) atmosphere, the camphoric acid salt (iii) (5 g) in absolute ethanol (20 g) is added. Then, isopropanol (25 g) is added and the reaction mixture is heated to reflux temperature of 90° C. (external temperature), 75° C. (internal temperature). The reaction mixture is refluxed for 30 minutes at an internal temperature of 75° C. The reaction mixture is stirred. The reaction mixture is then cooled over a period of 2 hours to an internal temperature of 0° C. Then the resulting suspension is heated to an internal temperature of 65° C. (in an ultrasonic bath) and then the reaction is seeded with the resolved camphoric acid salt (IV) (5 mg) in isopropanol (0.1 g). Then the reaction mixture is cooled to an internal temperature of 0 to 5° C. over a period of 15 minutes. Then, isopropanol (20 g) is added over a period of 30 minutes and the reaction mixture is stirred at an internal temperature of 0 to 5° C. Then, at an internal temperature of 0 to 5° C. further isopropanol is added (3×5 g). The reaction mixture is then filtered at an internal temperature of 0 to 5° C. (external temperature 0° C.) and dried under vacuum conditions at a temperature of 55° C.

### Example 4

### Synthesis of (2E)-N-[2-[2-[(2S)-1-Methyl-2-piperidinyl]ethyl]phenyl]-3-phenyl-2-propenamide

### (a) Free Base Generation

A 250-mL, 4-necked, round-bottomed flask, equipped with a mechanical stirrer, digital thermometer and nitrogen inlet-outlet, heating cooling bath, and addition funnel, is charged with 6.28 g (S)-2-[2-(1-methyl-2-piperidinyl)ethyl]-benzenamine (1R,3S)-(+)-camphoric acid salt (1:1) and 60 mL of isopropyl acetate. Stir the mixture at 20-25° C. under nitrogen and add a solution of 1.60 g of sodium hydroxide in 20 mL of water over a period of 5 min while maintaining an internal temperature at 20-25° C. Stir the suspension efficiently until all the solid dissolves (5 min). Separate the organic layer and save. Extract the aqueous layer with 20 mL of isopropyl acetate. Combine the organic layers and wash it with 20 mL of water. Separate the organic layer and concentrate it under vacuum (20-100 mbar) at an internal temperature at 20-40° C. (external temperature 30-60° C.) to obtain ˜65 mL of a solution of (S)-2-[2-(1-methyl-2-piperidinyl)ethyl]-benzenamine (containing 3.28 g of free base) in isopropyl acetate. Save this solution for the next step and store it under nitrogen.

### (b) Reaction

A 250-mL, 4-necked, round-bottomed flask, equipped with a mechanical stirrer, digital thermometer, nitrogen inlet-outlet, heating mantle, condenser, and addition funnel is charged with ˜65 mL of a solution of (S)-2-[2-(1-methyl-2-piperidinyl)ethyl]-benzenamine (containing 3.28 g of free base) in isopropyl acetate and 6.22 g of potassium carbonate. Stir the reaction mixture under nitrogen at an internal temperature at 23±3° C. to afford a suspension. Add 3.75 g of cinnamoyl chloride over a period of 5 min while maintaining an internal temperature at 23±3° C. to obtain a slurry. Heat the reaction mixture to an internal temperature at 85±5° C. (external temperature 90-100° C.) over a period of 30-60 min. Stir the reaction mixture at this temperature for an additional 2 h. Cool the reaction mixture to 23±3° C. over a period of 1 h. Add 50 mL of water. Stir the reaction mixture at 23±3° C. for 30-60 min to obtain a bi-phasic solution. Separate the organic layer. Add 80 mL of 0.5 N HCl solution over a period of 10 min while maintaining an internal temperature at 23±3° C. to afford a bi-phasic solution. Separate the aqueous layer. Add 60 mL of isopropyl acetate. Stir the reaction mixture and add a solution of 2.00 g of sodium hydroxide in 25 mL of water over a period of 10 min while maintaining an internal temperature at 23±3° C. to afford a bi-phasic solution. Separate the organic layer and save. Extract the aqueous layer with 60 mL of isopropyl acetate. Combine the organic layers and wash it with 40 mL of water. Separate the organic layer and concentrate it under vacuum (20-100 mbar) at an internal temperature at 20-40° C. (external temperature 30-60° C.) to obtain 22 mL (19.3 g) of a solution of (iii) in isopropyl acetate. Stir and heat the reaction mixture to an internal temperature at 85±5° C. (external temperature 90-100° C.) over a period of 30-60 min. Add 96 mL of hepatane over a period of 10 min while maintaining an internal temperature at 85±5° C. Stir and Cool the reaction mixture to 23±3° C. over a period of 1 h. Stir the resulting slurry at 23±3° C. for an additional 2 h. Collect the solid by filtration over a polypropylene filter paper in a Buchner funnel with suction. Wash the solid with a total of 28 mL of a mixture of isopropyl acetate and heptane (1/6) in two equal portions of 14 mL each. Dry the solid at 45-50° C. under vacuum (13-40 mbar) with nitrogen bleeding to obtain a constant weight (LOD<1%, 4 h) of 4.06 g of (2E)-N-[2-[2-[(2S)-1-methyl-2-piperidinyl]ethyl]phenyl]-3-phenyl-2-propenamide as an off white solid.

Theoretical Yield: 5.23 g

Yield: 77.6%

Purity: 99.8% (HPLC area %).

Enantiomeric purity: (R)-(iii) was not detected by Chiral HPLC.

### Example 5

### Alternative synthesis of (2E)-N-[2-[2-[(2S)-1-Methyl-2-piperidinyl]ethyl]phenyl]-3-phenyl-2-propenamide

### (a) Free Base Generation

In a 500 ml round bottomed flask equipped with a mechanical stirrer the resolved camphoric acid salt (IV) (20 g) in isopropyl acetate (120 g) is added at an internal temperature of 20 to 25° C. (external temperature 20° C.). Then, at an internal temperature of 25 to 30° C. (external temperature 20° C.) a solution of sodium hydroxide (38.24 g) in water (60 g) is added to the reaction mixture over a period of 5 minutes. The reaction mixture (suspension) is then stirred for a further 30 minutes. The resulting orange emulsion is then allowed to separate into a two-phase mixture and the water phase is removed. The organic phase is then subjected to a rotary evaporator and the isopropyl acetate is distilled at an internal temperature of 60° C. and under reduced pressure (250 mbar). Approximately 90 g of isopropyl acetate is distilled. Prior to distilling, the organic phase is a clear, bright orange colour and of a volume of approximately 160 ml (130 g),

### (b) Reaction

In a 1.5 l flask equipped with a mechanical stirrer and at an internal temperature of 35° C. (external temperature 38° C.) and under inert conditions (nitrogen) 2-butanone (160 g) and isopropyl acetate (20 g) is added to the reaction mixture of part (a). Then, at an internal temperature of 35° C. (external temperature of 38° C.) a solution of cinnamoyl chloride (8.9 g) in 2-butanone (20 g) is added drop wise. Then, the reaction mixture is treated with more 2-butanone (2×5 g). The resulting suspension is then stirred for 20 minutes at an internal temperature of 35° C. The pH of the mixture is between 6 and 8.

### (c) Resolution

The suspension of step (b) is then cooled to an internal temperature of 25° C. (external temperature 20° C.) and at the same time a mixture of water (200 g) and isopropyl acetate (60 g) is added. The reaction mixture is then stirred for a further 15 minutes at an internal temperature of 25° C. (external temperature 20° C.). The resulting two-phase reaction mixture is then separated and the water phase removed. The resulting yellow upper layer is then treated with 2.5 mol/l hydrochloric acid (200 g). The resulting two-phase mixture is then separated and the water phase is transferred into a 750 ml flask equipped with a mechanical stirrer. The organic phase is then washed with 2.5 mol/l hydrochloric acid (200 g) and the resulting two-phase mixture is separated and the water phase is added to the first water phase. The combined water phases are then treated with acetic acid (300 g) and sodium hydroxide (150 g) is added. The reaction mixture is then stirred at an internal temperature of 25 to 30° C. (external temperature 20° C.) for 15 minutes. The resulting two-phase reaction mixture is then separated.

### (d) Crystallization

The organic phase from the above reaction step (c) is reduced in volume on a rorary evaporator at an external temperature of 60° C. and at 250 mbar. Then, the reduced-volume reaction mixture is treated with isopropanol (60 g) and the resulting reaction mixture is reduced in volume on a rotary evaporator at an external temperature of 60° C. and under a vacuum of 150 mbar. Then, at an internal temperature of 50 to 55° C. (external temperature 60° C.) the reaction mixture is treated with water (20 g) and the resulting suspension is further treated with the product (iv) (10 mg) in isopropanol (0.01 g). The reaction mixture is then stirred for a further 15 minutes at an internal temperature of 50 to 55° C. (external temperature 60° C.). Then, further water is added over a period of 15 to 30 minutes and the reaction mixture is maintained at an internal temperature of 50 to 55° C. (external temperature 60° C.). Then, the resulting suspension is cooled to an internal temperature of 22 to 22° C. (external temperature 20° C. Then, the suspension is stirred for a further 30 minutes at an internal temperature of 22 to 22° C. (external temperature 20° C.) and the resulting solid is collected by filtration and washed with a mixture of water and isopropyl acetate (2×20 g), where the water:isopropyl acetate ratio is of 5:1 g/g. The resulting solid may then be dried under a vacuum at a temperature of 55° C.

Yield: 14.8 g (89.3% of theory).

mp: 127.3 to 130.2° C.

### Example 6

### Recrystallisation of (2E)-N-[2-[2-[(2S)-1-Methyl-2-piperidinyl]ethyl]phenyl]-3-phenyl-2-propenamide

(a) In a 200 ml round bottomed flask equipped with a magnetic stirrer, containing the product (iv) (15 g) is added isopropanol (25 g) and heptane (heptane fraction from petroleum having a boiling point of 65 to 100° C.) (25 g) is added. Then, the reaction mixture is heated to an internal temperature of 75° C. (external temperature 95° C.) and refluxed for approximately 30 minutes, whilst stirring. Then, the reaction mixture is filtered over a glass fibre filter at an internal temperature of 70 to 75° C. (external temperature 85° C.) in to a 350 ml flask equipped with a magnetic stirrer. Then, a mixture of isopropanol (5 g) and heptane (5 g) is added and the reaction mixture is heated to an internal temperature of 70° C. (external temperature 95° C.). Then, further heptane is added drop wise to the reaction mixture at an internal temperature of 65 to 75° C. (external temperature 75° C.).

### (b) Crystallization

The solution from step (a) is then cooled to an internal temperature of 40° C. (external temperature 40° C.) over a period of 15 minutes. The, at an internal temperature of 40° C., the solution is treated with a suspension of the recrystallized product (v) (11 mg) in heptane is added and the reaction mixture is stirred for 30 minutes at an internal temperature of 40° C. (external temperature 40 to 45° C.). Then, the reaction mixture is treated with some further heptane (15 g) at an internal temperature of 40° C. The resulting suspension is then cooled to an internal temperature of −10° C. (external temperature −10 to −15° C.) over a period of 30 minutes and then further stirred for a further hour. The reaction mixture is then filtered at an internal temperature of −10° C. (external temperature −10 to −15° C.) and the resulting solid may be washed in a mixture of isopropanol and heptane, where the isopropanol:heptane ratio is 1:1.5. The solid may be washed twice (2×11.25 g). The solid may then be dried in a vacuum at a temperature of 60° C.

Yield: 17.8 g (89% of theory)

mp: 127.4 to 132.0° C.

1: A process for providing the (S)-isomer (IV) of a compound (III) substantially separated from the (R)-isomer of compound (III): 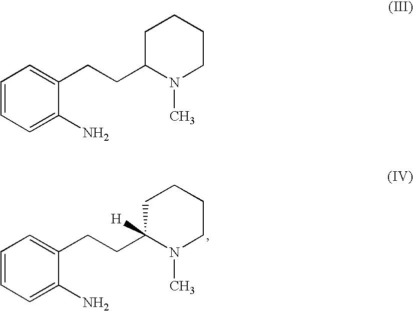
where the process comprises:
(a) providing compound (III) in the form of a salt of formula (II): 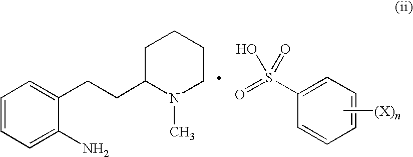
where X is an organic or inorganic moiety,
n is 0, 1, 2, 3 or 4; and
(b) resolving the isomers of compound (III) with a resolving agent.

2: The process of claim 1, wherein the resolving of the isomers comprises
(b1) converting the salt (ii) into its free base, namely compound (III); and
(b2) contacting the free base with the resolving agent.

3: The process of claim 2, wherein the free base is not isolated before being contacted with the resolving agent.

4: The process of claim 2, wherein the free base is isolated before being contacted with the resolving agent

5: The process of claim 1 wherein the resolving agent is an acid which, when contacted with the free base (III) forms a precipitate with one of the (R)- and (S)-isomers of the free base (III).

6: The process of claim 5, wherein the (S)-isomer (IV) of the free base (III) forms the precipitate with the resolving agent.

7: The process of claim 5, wherein the resolving agent is a camphoric acid.

8: The process of claim 5, wherein the resolving agent is

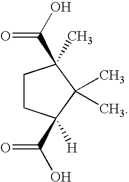

9: The process of claim 1, which further comprises converting the resolved isomer (IV) and/or a salt thereof into a compound of formula (I) or a pharmaceutically acceptable salt or prodrug thereof:

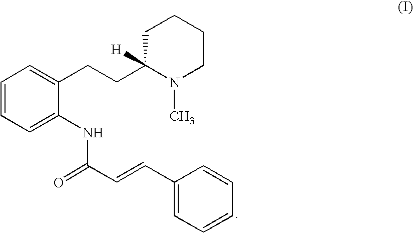

10: The process of claim 9, wherein a salt of isomer (IV) is converted into its free base before converting to compound (I).

11: The process of claim 9, wherein said converting process comprises reacting isomer (IV) with cinnamoyl chloride.

12: The process of claim 11, wherein the reaction is performed in the presence of a carbonate.

13: The process of claim 1, further comprising recrystallising the product.

14: The process of claim 9, wherein the process additionally comprises converting the product thereof into a pharmaceutical formulation.
